(19) United States
(12) Patent Application Publication
(10) Pub. No.: US 2022/0088042 A1
(43) Pub. Date: Mar. 24, 2022

(54) COMBINATION THERAPY INCLUDING SAPC-DOPS FOR THE TREATMENT OF PANCREATIC CANCER

(71) Applicants: Bexion Pharmaceuticals, Inc., Covington, KY (US); University of Cincinnati, Cincinnati, OH (US)

(72) Inventors: Xiaoyang Qi, Cincinnati, OH (US); Olugbenga Olowokure, Cincinnati, OH (US); Ray Takigiku, Covington, KY (US)

(21) Appl. No.: 17/327,863

(22) Filed: May 24, 2021

Related U.S. Application Data
(63) Continuation of application No. 15/819,880, filed on Nov. 21, 2017, now abandoned.
(60) Provisional application No. 62/424,573, filed on Nov. 21, 2016.

(51) Int. Cl. A61K 31/685; A61K 38/17; A61K 31/7068; A61K 45/06; A61K 9/00; A61P 35/00; A61K 31/337; A61K 9/127; A61K 9/107; A61K 31/513; A61K 47/24; A61K 31/695; A61K 38/16; A61K 31/4745; A61K 31/555; A61K 31/519

(52) U.S. Cl. CPC A61K 31/685; A61K 2300/00; A61K 31/7068; A61K 45/06; A61K 9/0019; A61P 35/00; A61K 31/337; A61K 9/127; A61K 9/1075; A61K 31/513; A61K 47/24; A61K 31/695; A61K 38/16; A61K 31/4745; A61K 31/555; A61K 31/519; A61K 38/17

(57) ABSTRACT

Disclosed herein, according to the present invention, are methods of treating pancreatic cancer comprising administering a first pharmaceutical composition comprising Saposin C and dioleoylphosphatidylserine (SapC-DOPS) and administering a second pharmaceutical composition comprising an anti-neoplastic agent. Optionally, additional pharmaceutical compositions may be administered. Also disclosed are methods of inhibiting tumor growth. Also disclosed are kits for the treatment of pancreatic cancer comprising at least two pharmaceutical compositions, wherein a first pharmaceutical composition comprises SapC-DOPS and wherein a second pharmaceutical composition comprises a first antineoplastic agent. Also disclosed herein are combination therapeutics comprising a first pharmaceutical composition comprising SapC-DOPS and at least a second pharmaceutical composition comprising an antineoplastic agent, wherein the first and second pharmaceutical compositions are formulated separately to be used in the form of a kit where they are present together.

### CROSS-REFERENCE TO RELATED APPLICATIONS

This application is a continuation of U.S. patent application Ser. No. 15/819,880, filed on Nov. 21, 2017, which claims priority to and the benefit of U.S. Provisional Patent Application Ser. No. 62/424,573, filed on Nov. 21, 2016, now expired, which applications are hereby incorporated herein by reference in their entirety for all purposes.

### TECHNICAL FIELD

The present disclosure relates to the field of anti-cancer therapeutics and more particularly to methods for the treatment of pancreatic cancer.

### BACKGROUND

Pancreatic cancer is the fourth leading cause of cancer deaths, with a 5-year survival of less than 5%. It is usually asymptomatic in the early stages, while frequently invading regional lymph nodes and liver, and less often the lungs and visceral organs. Current multi-modal strategies, including surgery, chemotherapy, and radiation therapy, have failed to improve long term survival. The current standard of treatment, the nucleoside analog gemcitabine, prolongs survival by only several months. Despite exhaustive efforts to map the genetic alterations associated with pancreatic cancer growth, few promising drug targets have been reported, and new, effective treatments are urgently needed. Experimental therapeutic strategies include small and large molecule inhibitors of oncogenic pathways, anti-angiogenic agents, vaccination/immunotherapy, gene therapies, and many others, but no clearly superior therapies have emerged.

Clearly additional agents and treatment options are needed in the battle against pancreatic cancer.

### SUMMARY

Accordingly, a novel combination therapy is provided for the treatment of cancer, including pancreatic cancer. SapC-DOPS is a novel anticancer nanovesicle that targets surface exposed phosphatidylserine (PS) in pancreatic cancer cells. Gemcitabine, a first-line chemotherapeutic drug for human pancreatic cancer, synergistically potentiated the anticancer effects of SapC-DOPS by elevating PS exposure in the surface of pancreatic tumor cells. Combination treatments comprising administration with gemcitabine prior to or concurrent with administration of SapC-DOPS is surprisingly efficacious in causing neoplastic cell apoptosis, inhibiting tumor growth, and shrinking or eradicating existing tumors.

Disclosed herein, according to the present invention, is a method of treating pancreatic cancer comprising administering a first pharmaceutical composition comprising Saposin C and dioleoylphosphatidylserine (SapC-DOPS) and administering a second pharmaceutical composition comprising an anti-neoplastic agent. Optionally, additional pharmaceutical compositions may be administered.

Also disclosed herein, according to the present invention, is a method of inhibiting tumor growth comprising administering a first composition comprising SapC-DOPS and administering a second pharmaceutical composition comprising an antineoplastic agent. Optionally, additional pharmaceutical compositions may be administered.

Also disclosed herein, according to the present invention, is a kit for the treatment of pancreatic cancer comprising at least two pharmaceutical compositions, wherein a first pharmaceutical composition comprises SapC-DOPS and wherein a second pharmaceutical composition comprises a first antineoplastic agent.

Also disclosed herein, according to the present invention, is a combination therapeutic comprising a first pharmaceutical composition comprising SapC-DOPS and at least a second pharmaceutical composition comprising an antineoplastic agent, wherein the first and second pharmaceutical compositions are formulated separately to be used in the form of a kit where they are present together.

### BRIEF DESCRIPTION OF THE DRAWINGS

FIGS. 1A, 1B; SapC-DOPS selectively kills human pancreatic tumor cells. FIG. 1A) Three pancreatic tumor cell lines, as well as non-transformed human pancreatic ductal epithelial cells (HPDE) were exposed to SapC-DOPS (0.14 mg SapC) or vehicle (PBS) and viability was assayed 72 h later. Note that non-tumoral HPDE cells were not affected. FIG. 1B) Absence of effect of DOPS liposomes on both tumor and normal pancreatic ductal cells.

FIGS. 2A-2C; In vivo targeting and antitumor activity of SapC-DOPS in pancreatic cancer. FIG. 2A) Subcutaneous pancreatic tumors created using CFPAC-1-Luc3 cells pretreated with or without PS-specific binding proteins [Lactadherin-C2 (upper panel] and Beta-GP-1 [lower panel])] display bioluminescence on live imaging (left). 24 h after i.v. injection with fluorescently labeled SapC-DOPS nanovesicles (SapC-DOPS-CVM; right), tumors that were not pretreated demonstrated fluorescence, while the tumors that had been pretreated were not targeted. FIG. 2B) Whole body imaging showing targeting of both the primary pancreatic tumor (CFPAC-1-Luc3 cells) and a lung metastasis after an i.v injection with SapC-DOPS-CVM (left). Tumor presence was confirmed by bioluminescence (right). FIG. 2C) Kaplan-Meier survival curve of mice bearing human pancreatic tumors—as in (B)—and treated with PBS (control) or SapC-DOPS. Tumor resolution was observed in 4/6 mice treated with SapC-DOPS.

FIG. 3. GEM exposure triggers PS externalization in pancreatic tumor cells. AsPC-1 (A) and Mia-PaCa-2 (B) cells were treated with varying doses of GEM for 24 h, TUNEL apoptosis stain was performed after surface PS staining with annexin V-APC. The overlay histograms show TUNEL-negative (non-apoptotic) cells. TUNEL positive [AsPC-1: CTL=0.3; GEM 10 nM=2.5; GEM 100 nM=4.6]; [MIA-PaCa-2: CTL=3.6; GEM 1 μM=21].

FIG. 4. Synergistic antitumor effect of GEM plus SapC-DOPS on cultured pancreatic tumor cells. Combination treatment with SapC-DOPS plus GEM elicits marked, synergistic cell death on cultured human MiaPaCa-2 cells. Cells were exposed (72 h) to vehicle, GEM (50 nM), SapC-DOPS (4-uM SapC) or SapC-DOPS plus GEM.

FIGS. 5A-5C; Enhanced antitumor effects of SapC-DOPS plus GEM against pancreatic cancer. FIG. 5A) Tumor size chart from mice bearing subcutaneous pancreatic tumor xenografts (Mia-PaCa-2 cells). After tumor mean volume reached 100 mm³, mice (12/group) were treated with Saline (control), GEM (40 mg/kg/i.p.), SapC-DOPS (4.9 mg/kg/i.v.) or SapC-DOPS plus GEM. Injections were applied starting on day 26 and every 3 days thereafter until sacrifice. FIG. 5B) Photograph of the excised tumors (day 39). Combination treatment with SapC-DOPS and GEM effectively suppressed tumor growth. FIG. 5C) Tumor weight at sacrifice.

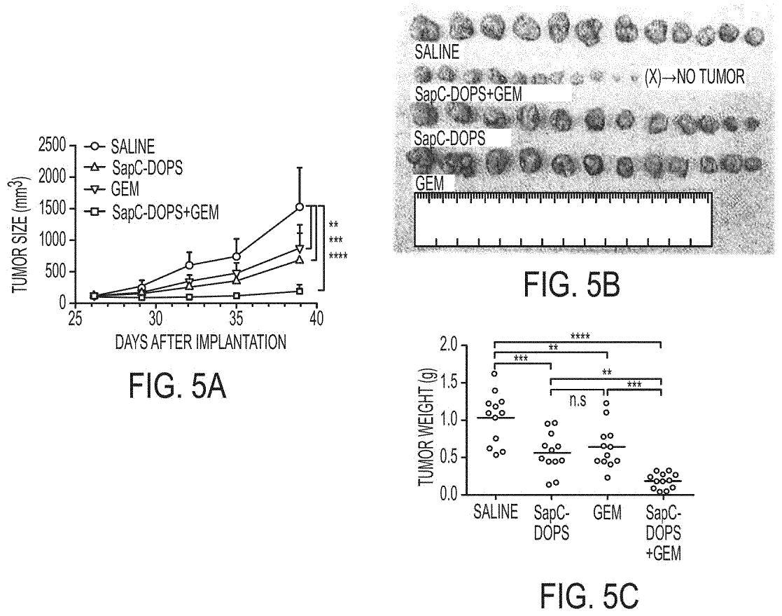

FIG. 6. Effect of treatment on tumor weight. Tumor weight of tumors at sacrifice (day 35) following inoculation of mice on day 1 with subcutaneous pancreatic tumor xenografts (Mia-PaCa-2 cells). Mice were inoculated on day 1, and on day 5 mice were injected with VEH (control), GEM (40 mg/kg/i.p.), or SapC-DOPS plus GEM (7 mg/kg/i.v. SapC-DOPS plus 40 mg/kg/i.p. GEM).

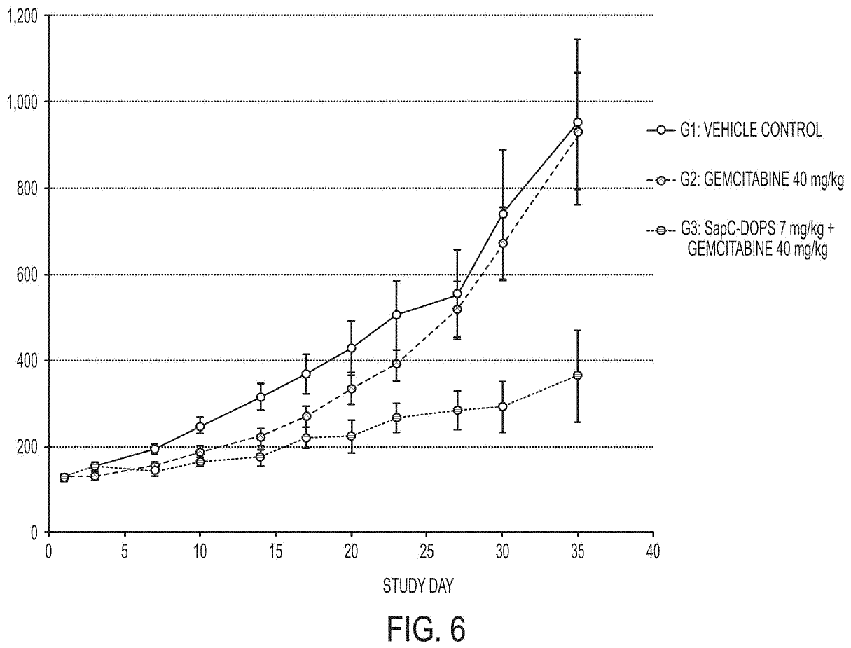

FIG. 7. Effect of treatment on body weight. Percent change in body weight of mice at sacrifice (day 35) following inoculation of mice on day 1 with subcutaneous pancreatic tumor xenografts (Mia-PaCa-2 cells). Mice were inoculated on day 1, and on day 5 mice were injected with VEH (control), GEM (40 mg/kg/i.p.), or SapC-DOPS plus GEM (7 mg/kg/i.v. SapC-DOPS plus 40 mg/kg/i.p. GEM).

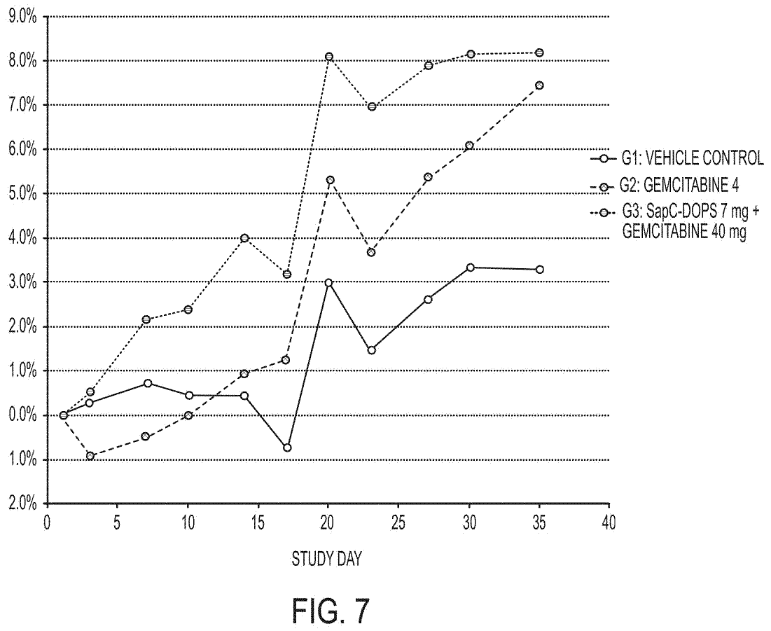

FIG. 8. Cell viability of mouse pancreatic cancer cells (p53.2.1.1) by MTT assay following treatment with SapC-DOPS (20 μM), 25 nM Abraxane® plus 25 nM gemcitabine, or SapC-DOPS (20 μM) plus 25 nM Abraxane® plus 25 nM gemcitabine. Combination treatment with SapC-DOPS plus Abraxane® plus gemcitabine elicited a marked, synergistic cell viability on cultured mouse cells.

### DETAILED DESCRIPTION

For purposes of the following detailed description, it is to be understood that the invention may assume various alternative variations and step sequences, except where expressly specified to the contrary. Moreover, other than in any operating examples, or where otherwise indicated, all numbers such as those expressing values, amounts, percentages, ranges, subranges and fractions may be read as if prefaced by the word “about,” even if the term does not expressly appear. Accordingly, unless indicated to the contrary, the numerical parameters set forth in the following specification and attached claims are approximations that may vary depending upon the desired results to be obtained by the present invention. At the very least, and not as an attempt to limit the application of the doctrine of equivalents to the scope of the claims, each numerical parameter should at least be construed in light of the number of reported significant digits and by applying ordinary rounding techniques. Where a closed or open-ended numerical range is described herein, all numbers, values, amounts, percentages, subranges and fractions within or encompassed by the numerical range are to be considered as being specifically included in and belonging to the original disclosure of this application as if these numbers, values, amounts, percentages, subranges and fractions had been explicitly written out in their entirety.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

As used herein, unless indicated otherwise, a plural term can encompass its singular counterpart and vice versa, unless indicated otherwise. For example, although reference is made herein to “a” composition, a combination (i.e., a plurality) of these components can be used. In addition, in this application, the use of “or” means “and/or” unless specifically stated otherwise, even though “and/or” may be explicitly used in certain instances.

As used herein, “including,” “containing” and like terms are understood in the context of this application to be synonymous with “comprising” and are therefore open-ended and do not exclude the presence of additional undescribed and/or unrecited elements, materials, ingredients and/or method steps.

As used herein, “consisting of” is understood in the context of this application to exclude the presence of any unspecified element, ingredient and/or method step.

As used herein, “consisting essentially of” is understood in the context of this application to include the specified elements, materials, ingredients and/or method steps “and those that do not materially affect the basic and novel characteristic(s)” of what is being described.

As used herein, “patient” or “subject” refers to animals, including mammals, including humans.

As used herein, “pharmaceutical composition” refers to any chemical or biological composition, material, agent or the like that is capable of inducing a therapeutic effect when properly administered to a subject, including the composition, material, agent or the like in an inactive form and active metabolites thereof, where such active metabolites may be formed in vivo.

As used herein, “combination therapeutic” refers to at least two pharmaceutical compositions, formulated separately, and administered together either sequentially or contemporaneously, to act as an antineoplastic agent.

As used herein, the term “Saposin C” or “SapC” refers to an 80-amino acid membrane associated protein (SEQ ID NO. 1) (naturally occurring and synthetic) that distributes in lysosomes of all cell types, and also including homologues thereof, wherein the homologue possesses at least 75% sequence homology, due to degeneracy of the genetic code which encodes for SapC, and polypeptides and peptide analogues possessing similar biological activity as SapC.

As used herein, the term “DOPS” refers to dioleoylphosphatidylserine, a phospholipid located on cell membranes.

As used herein, the term “SapC-DOPS” refers to the combination of SapC and DOPS.

As used herein, when a dosage of SapC-DOPS is reported, the dosage refers to the dose of SapC. For example, a dosage of SapC-DOPS of 2.4 mg/kg refers to 2.4 mg/kg of SapC.

As used herein, the term “anti-neoplastic agent” refers to an agent that prevents or inhibits the development or growth of a neoplasm.

As used herein, the “neoplasm” refers to a new and abnormal growth of tissue, including cancer and metastatic cancer.

As used herein, the term “sequentially” refers to a treatment protocol in which administration of a first treatment, such as administration of a pharmaceutical composition, follows administration of a second treatment, such as administration of a secondphartnaceutical composition.

As used herein, the term “contemporaneously” refers to administration of a first treatment, such as administration of a first pharmaceutical composition, and administration of a second treatment, such as administration of a second pharmaceutical composition, wherein the first and second treatments are separate and are administered at substantially the same time. Where the treatments comprise administration of first and second pharmaceutical compositions, the first and second pharmaceutical compositions are separate (i.e., active ingredients are not in the same composition) and are administered at substantially the same time.

Disclosed herein, according to the present invention, are compositions and methods useful for treating cancer, such as pancreatic cancer. The compositions and methods of the present invention include first-line and second-line combination therapies, and may be used to treat resectable pancreatic cancers, locally advanced unresectable pancreatic cancers, and metastatic cancers. As described in more detail below, the present invention makes use of a combination treatment in which a first pharmaceutical composition and a second composition are administered either sequentially or contemporaneously to treat pancreatic cancer and/or to inhibit tumor growth. The first composition may comprise, or consist essentially of, or consist of, Saposin C and dioleoylphosphatidylserine (SapC-DOPS) and the second composition may comprise, or consist essentially of, or consist of, an anti-neoplastic agent. As described in more detail below, the present invention is based on the surprising discovery that some antineoplastic agents employed in standard chemotherapy treatments for pancreatic tumors and other cancers potentiate the anti-tumor actions of SapC-DOPS. Without being bound by theory, it is posited that certain cytotoxic antineoplastic agents increase the levels of surface phosphatidylserine, thus providing a more salient target for the action of SapC-DOPS. The cytotoxic agents may increase PS via apoptosis; however, the more resilient tumor cells may also increase their surface PS in an effort to increase the immunosuppressive environment, and counteract the cytotoxic agent effectiveness. As such a combined therapy may provide a greater synergistic effect.

Pharmaceutical Compositions

SapC-DOPS

Phosphatidylserine (PS) is an anionic phospholipid with important structural and signaling properties. It normally localizes in the internal leaflet of the lipid bilayer in the plasma membrane of animal cells. Notably, viable cancer cells and tumor-associated vascular cells usually present elevated levels of PS on the external surface of their membranes. This may imply that high external PS confers an adaptive advantage to cancer cells. There is also evidence that tumor immunity and metastatic potential may be counteracted and favored, respectively, by increased surface PS levels. PS is a unique therapeutic target for SapC-DOPS nanovesicles in pancreatic cancer treatment. SapC-DOPS nanovesicle is a new biologic anticancer agent that contains a human protein, saposin C (SapC), associated with lipophilic nanovesicles composed of dioleoylphosphatidylserine (DOPS). SapC is a naturally occurring membrane protein that binds PS with high affinity and activates lysosomal enzyme, leading to ceramide production and apoptotic cancer cell death. By targeting PS-rich domains on neoplastic cell membranes, SapC-DOPS has been shown to selectively kill tumor cells using both in vivo and in vitro models of pancreatic cancer without overt off-target toxicity to normal cells and tissues. Unlike most standard therapies, SapC-DOPS exerts direct cytotoxicity in a variety of cancer cells with diverse genetic profiles. Furthermore, tumor growth is effectively delayed or blunted, with little or no impact on normal cells and organs, revealing an essentially complete lack of toxicity. In summary, SapC-DOPS is a first-in-class, biologically active anticancer agent that comprises naturally occurring molecules and effectively targets and eradicates preclinical tumors through a PS-dependent mechanism. Remarkably, elevated surface PS exposure is a common feature of many different tumor cells and their associated vasculature, and can be considered a pan-tumoral marker. SapC-DOPS targets a ubiquitous, cell surface-exposed tumor marker, offering a unique approach for both diagnosing and treating pancreatic cancer and potentially other types of cancer.

Thus, according to the present invention, the first pharmaceutical composition may comprise an amount of SapC (SEQ. ID. NO. 1) and an amount of DOPS,

SEQ ID NO 1:
Ser-Asp-Val-Tyr-Cys-Glu-val-Cys-Glu-Phe-Leu-Val-
Lys-Glu-Val-Thr-Lys-Leu-Ile-Asp-Asn-Asn-Lys-Thr-
Glu-Lys-Glu-Ile-Leu-Asp-Ala-Phe-Asp-Lys-Met-Cys-
Ser-Lys-Leu-Pro-Lys-Ser-Leu-Ser-Glu-Glu-Cys-Gln-
Glu-Val-Val-Asp-Thr-Tyr-Gly-Ser-Ser-Ile-Leu-Ser-
Ile-Leu-Leu-Glu-Glu-Val-Ser-Pro-Glu-Leu-Val-Cys-
Ser-Met-Leu-His-Leu-Cys-Ser-Gly.

Alternatively, an anionic phospholipid or phospholipid with an overall negative charge may be used instead of DOPS. SapC-DOPS is described in U.S. Pat. No. 7,834,147, incorporated herein by reference. The molar ratio of SapC-DOPS may be 1:1 to 50:1, such as 1:7 to 1:25. The SapC and DOPS combined may form a nanovesicle. Nanovesicles may be 10 nm to 800 nm, such as 40 nm to 200 nm. The first pharmaceutical composition may have apH of 5 to 8, such as 7 to 7.4.

The first composition may further comprise a pharmaceutically acceptable carrier. As used herein, the term “pharmaceutically acceptable carrier” includes any and all solvents, diluents, or other liquid vehicle, dispersion or suspension aids, surface active agents, isotonic agents, thickening or emulsifying agents, stabilizers, preservatives, solid binders, lubricants, and the like, as suited to the particular dosage form desired. Remington's Pharmaceutical Sciences Ed. by Gennaro, Mack Publishing, Easton, Pa., 1995 provides various carriers used in formulating pharmaceutical compositions and known techniques for the preparation thereof. Examples of pharmaceutically acceptable carriers are sugars such as monosaccharides, disaccharides, and the like, excipients such as cocoa butter and waxes; oils such as peanut oil, cottonseed oil, safflower oil, sesame oil, olive oil, corn oil, and soybean oil; glycols such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; phosphate buffer solutions; other non-toxic compatible lubricants such as sodium lauryl sulfate and magnesium stearate; coloring agents, releasing agents, coating agents, preservatives and antioxidants according to the judgment of the formulator.

Antineoplastic Agents

According to the present invention, the second composition may comprise at least one antineoplastic agent. The second composition may further comprise a pharmaceutically acceptable carrier, as described above.

Gemcitabine

The antineoplastic agent of the second composition of the present invention may comprise gemcitabine. Gemcitabine HCL (GEM) is a known chemotherapy drug for cancer treatment. GEM and its preparation are described in U.S. Pat. No. 4,808,614, issued Feb. 28, 1989 to Larry Hertel (see 1:54-19:55, incorporated herein by reference). The chemical formula of GEM is C₉H₁₁F₂N₃O₄ and the chemical structure is

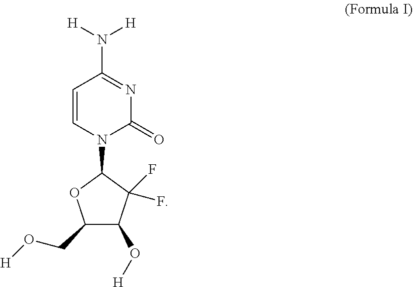 [SMILES: Nc1ccn([C@@H]2O[C@H](CO)[C@H](O)C2(F)F)c(=O)n1]

The use of GEM to treat cancer is known and is described in U.S. Pat. No. 5,464,826, issued on Nov. 7, 1995 to Gerald B. Grindey et al. (see 15:53-23:40, incorporated herein by reference) and was originally distributed by Eli Lilly and Company under the brand name Gemzar®.

For example, gemcitabine is a nucleoside analog that is utilized as a first-line treatment for many tumor types, including resectable pancreatic tumors and bladder tumors. As with fluorouracil and other analogues of pyrimidines, the triphosphate analogue of gemcitabine replaces one of the building blocks of nucleic acids, in this case cytidine, during DNA replication. The process arrests tumor growth, as only one additional nucleoside can be attached to the “faulty” nucleoside, resulting in apoptosis. Another target of gemcitabine is the enzyme ribonucleotide reductase (RNR). The diphosphate analogue binds to RNR active site and inactivates the enzyme irreversibly. Once RNR is inhibited, the cell cannot produce the deoxyribonucleotides required for DNA replication and repair, and cell apoptosis is induced. The present invention is based on the surprising discovery that some antineoplastic agents employed in standard chemotherapy treatments for pancreatic tumors and other cancers potentiate the anti-tumor actions of SapC-DOPS. Without being bound by theory, it is posited that certain cytotoxic antineoplastic agents increase the levels of surface PS, thus providing a more salient target for the action of SapC-DOPS.

Pharmaceutical compositions comprising GEM may be formulated and administered according to the methods of the present invention either prior to, sequentially, or contemporaneously with SapC-DOPS in the therapeutically effective amounts or dosages described herein.

Paclitaxel

The antineoplastic agent of the second composition of the present invention may comprise paclitaxel, a well-known chemotherapeutic drug that has significant antineoplastic effects. Paclitaxel is known in both its solvent-borne form (distributed by Bristol-Myers Squibb Company as Taxol®) and as nab-paclitaxel, in which paclitaxel is bound to albumin-nanoparticles (distributed by Celgene Corporation as Abraxane®). Paclitaxel is utilized as a first-line and second-line treatment for many cancer types, including ovarian, breast, lung, pancreatic, bladder, prostate, non-small cell lung cancers, melanoma, esophageal, solid cancers, and Kaposi sarcoma. Paclitaxel targets tubulin to prevent the normal breakdown of microtubules during cell division, thereby blocking the progression of mitosis. Paclitaxel has the chemical formula C₄₇H₅₁NO₁₄ and is described in U.S. Pat. No. 7,758,891, issued on Jun. 20, 2010 to Neil P. Desai et al. (see 5:25-21:33, incorporated herein by reference). Nab-paclitaxel is preferred treatment for pancreatic cancer. The structure of Taxol® is shown in Wani M C, Taylor H L, Wall M E, Coggon P, McPhail A T. Plant antitumor agents. VI. The isolation and structure of taxol, a novel antileukemic and antitumor agent from Taxus brevifolia. J Am Chem Soc. 1971 May 5; 93(9):2325-7, incorporated herein by reference.

### 5′-Fluorouracil-Based Chemotherapy (Folfirinox)

Folfirinox is a known combination chemotherapy regimen for treatment of advanced pancreatic cancer which includes the administration of four drugs per treatment cycle: folinic acid (leucovorin), a vitamin B derivative, fluorouracil (5-FU), a pyrimidine analog, irinotecan, a topoisomerase inhibitor, and oxaliplatin, a platinum-based antineoplastic agent.

Therapeutically Effective

As described above, the methods of treating pancreatic cancer and of inhibiting tumor growth provided herein comprise administering a therapeutically effective amount of a first pharmaceutical composition having as an active agent SapC-DOPS and a therapeutically effective amount of a second pharmaceutical composition having as an active agent an anti-neoplastic agent, to a subject in need thereof, in such amounts and for such time as is necessary to achieve the desired result. According to the invention, additional third, fourth, fifth, etc. compositions each comprising a different anti-neoplastic agent also may be administered to the subject sequentially or contemporaneously with the first and/or second, third, fourth, fifth, etc. compositions. As used herein, the first, second, third, etc. pharmaceutical compositions are not meant to denote the order of administration, and such pharmaceutical compositions may be administered contemporaneously and/or sequentially in any order in the treatment protocol.

The compositions, according to the method of the present invention, may be administered using an amount, such as a therapeutically effective dose, and a route of administration effective for contacting normal cells, cancer cells or tumor cells. As used herein, the terms “therapeutically effective dose” and “amount effective for treating cancer, cancer cells, or tumors,” refer to that amount of active agent that modulates or ameliorates the symptoms or condition of a cancer, tumor, or neoplastic disease, e.g., prevents or reduces viability of the cancer cells and can include a single treatment or a series of treatments. A therapeutically effective dose may increase or decrease over the course of treatment. Therapeutic efficacy and toxicity of active agents may be determined by standard pharmaceutical procedures in cell cultures or experimental animals, e.g., ED50 (the dose is therapeutically effective in 50%© of the population) and LD50 (the dose is lethal to 50% of the population). The dose ratio of toxic to therapeutic effects is the therapeutic index, and it is expressed as the ratio, LD50/ED50. According to the present invention, pharmaceutical compositions may exhibit large therapeutic indices.

The skilled artisan understands that various factors influence the dosage required to treat a patient effectively, and that accordingly the dosage and administration may be chosen by the attending physician in view of the patient to be treated and may be adjusted for sufficient levels of the active agent(s) or to maintain the desired effect. Additional factors that may be taken into account include the severity of the disease state, e.g., intermediate or advanced stage of disease; age, weight, gender and overall health of the patient; diet, time and frequency of administration; size of the cancer or tumor; route of administration; drug combinations; reaction sensitivities; prior treatments; and tolerance/response to therapy. Pharmaceutical compositions may be administered, for example, 30 minutes, hourly or daily; multiple times per day; weekly, multiple times per week; bi-weekly; monthly; and the like, depending on the half-life and clearance rate of the particular composition.

The active agents of the invention may be used to treat any of the diseases, disorders, or the like disclosed herein and may be administered as a therapeutically effective dose appropriate for the patient to be treated. As described above, the therapeutic dose of the compositions of the present invention may be decided by the attending physician within the scope of sound medical judgment and experience. For the active agent, the therapeutically effective dose may be estimated initially in cell culture assays or in animal models such as mice, rats, rabbits, dogs, or pigs. Animal cell models may be used to achieve or determine a desirable concentration and total dosing range and route of administration, which may be used to determine a useful range of dosage and routes for administration in humans. Further, clinical studies and individual patient response may determine the recommended therapeutic dose.

A therapeutically effective dose of the first pharmaceutical composition comprising SapC-DOPS may ordinarily be administered at a dosage level of 0.5 mg SapC/kg of body weight to 7.0 mg SapC/kg of body weight per dose, such as 0.7 mg SapC/kg of body weight to 4.8 mg SapC/kg of body weight per dose.

A therapeutically effective dose of the second pharmaceutical composition comprising gemcitabine may ordinarily be administered at a dosage level of 800 mg/m² to 1250 mg/m², such as 1000 mg/m².

A therapeutically effective dose of the second pharmaceutical composition comprising paclitaxel may ordinarily be administered at a dosage level of 90 mg/m² to 250 mg/m², such as 125 mg/m² to 175 mg/m².

A therapeutically effective dose of the second pharmaceutical composition comprising nab-paclitaxel may ordinarily be administered at a dosage level of 100 mg/m² to 150 mg/m², such as 100 mg/m² to 125 mg/m².

A therapeutically effective dose of the second pharmaceutical composition comprising 5′-fluorouracil may ordinarily be administered at a dosage level of 500 mg/m² to 2000 mg/m² i.v and/or 300-500 mg/m² by i.v. bolus. Sequentially or contemporaneously with each dose of 5′-fluorouracil, one or more of the following may be administered to the patient, at the recommended dosage levels: irinotecan, ordinarily administered at a dosage level of 70 mg/m² to 180 mg/m²; oxaliplatin, ordinarily administered at a dosage level of 65 mg/kg to 85 mg/m²; and/or leucovorin, ordinarily administered at a dosage level of 200 mg/m² to 400 mg/m²,

Administration of Pharmaceutical Compositions

As described above, the methods described herein generally include the administration of a first pharmaceutical composition comprising SapC-DOPS and the administration of a second, separate pharmaceutical composition comprising an antineoplastic agent. As described above, additional pharmaceutical compositions comprising, consisting essentially of, or consisting of an antineoplastic agent also may be included in the method of the present invention. The first and second pharmaceutical compositions may be administered contemporaneously. Alternatively, the first and second pharmaceutical compositions may be administered sequentially, such that administration of the first pharmaceutical composition is followed by administration of the second pharmaceutical composition, or vice versa. When the first and second compositions are administered sequentially, the method may comprise waiting a period of time between the administration of the pharmaceutical compositions. According to the present invention, the administering of the pharmaceutical compositions, whether contemporaneous or sequential, may be given over the course of at last two cycles.

The combination therapies described herein have unexpectedly resulted in a synergistic therapy for the treatment of cancers and inhibition of tumor growth with methods that are lower toxicity, require lower doses of toxic compounds than conventional cancer or tumor treatments, and provide improved patient tolerance and response. A pharmaceutical composition of the present invention may be formulated in such a manner as to be administered via an intended route, such as intravenous, intradermal, subcutaneous, oral, intramuscular, subcutaneous, intratumor, transdermal, transmucosal, intraperitoneal, and rectal administration, and may include a pharmaceutically acceptable carrier (described herein) to form a solution, dispersion, emulsion, microemulsion, suspension, syrup, elixir or the like. According to the invention, the pharmaceutical composition may be suitable for bolus administration, such as a bolus intravenous infusion. pH adjusters (i.e., acids, or bases) may be included to adjust pH to the appropriate level, and/or antibacterial and antifungal agents may be included to prevent the action of microorganisms, Pharmaceutical compositions also may include formulations that control or slow release of the agent from the body. In some instances, the pharmaceutical composition may be included in a dispenser, such as a syringe, dosing vial, and the like.

Pharmaceutical compositions suitable for injection may include the active agent and a pharmaceutically acceptable carrier for formation of a sterile solution, dispersion, and the like, and may have a viscosity appropriate for injection.

Pharmaceutical compositions suitable for oral administration often include an inert diluent or carrier and may be enclosed in gelatinous capsules or compressed into tablets that also contain binders, excipients, lubricants, flavoring agents, and the like, may be in liquid form such that the materials may be swallowed or expectorated, or may be in aerosol form such that the composition may be expelled from a pressurized container.

Pharmaceutical compositions suitable for transdermal or transmucosal administration may include materials suitable for the formation of patches, ointments, gels, creams, salves, sprays, suppositories, and the like.

According to the present invention, the methods may further comprise administering to the subject a therapy sequentially or contemporaneously with the administering of the first and second pharmaceutical compositions. For example, the therapy may comprise surgery, radiotherapy, and/or chemotherapy. The therapy also may comprise administration of antibiotics, vitamins and other supplements, appetite stimulants, antiemetics, and other agents to maintain or improve the subject's general overall health and/or tolerance to treatment.

In each of the treatment protocols described herein, the first, second, and third pharmaceutical compositions are not meant to denote the order of administration, and the first, second, third, etc. pharmaceutical compositions may be administered contemporaneously and/or sequentially in any order in the treatment protocol. There may be periods of time between administering each of the pharmaceutical composition in the sequence.

Each protocol described below comprises one cycle, and each protocol may be administered to a patient for at least one cycle, such as at least two cycles, such as at least three cycles, such as at least four cycles, such as at least five cycles, such as at least six cycles. For each protocol described herein below, the therapeutic dose of the pharmaceutical compositions described are exemplary and doses may be decided and/or adjusted by the attending physician within the scope of sound medical judgment and experience. Although the examples herein may describe the administration of SapC-DOPS prior to administration of an antineoplastic agent, an antineoplastic agent, such as gemcitabine, may be administered to a patient for a period of time, such as one week, prior to cycles of sequential or contemporaneous administration with SapC-DOPS.

In an example, the present invention may be provided as an adjuvant therapy and may comprise, for example, administering a first pharmaceutical composition comprising SapC-DOPS and a second pharmaceutical composition comprising gemcitabine to a patient, either sequentially or contemporaneously. Each cycle may comprise the administration of the first and the second pharmaceutical compositions at least one time every week for three weeks with one week off on week four for a number of cycles, such as 6 cycles. Each cycle may comprise weekly administration of the first composition comprising SapC-DOPS at the doses provided herein or as determined by a treating physician, for example at a dose of 2.4 mg/kg, and sequential or contemporaneous administration of the second composition comprising gemcitabine at the doses provided herein or as determined by a treating physician, for example at a dose of 1000 mg/m². Each cycle may comprise administering SapC-DOPS more than once per week, such as three times per week or daily, and administering gemcitabine one time per week, such as on Day 1.

In an example, the present invention may be provided as an adjuvant chemotherapy together with chemoradiation and may comprise, for example, administering a first pharmaceutical composition comprising SapC-DOPS and a second pharmaceutical composition comprising gemcitabine to a patient, either sequentially or contemporaneously. Each cycle may comprise at least once weekly administration of the first composition comprising SapC-DOPS and sequential or contemporaneous administration of the second composition comprising gemcitabine, and sequential or contemporaneous administration of chemoradiation, such as administration of 5′-fluorouracil at a recommended dose, such as 250 mg/m²/day continuous IV infusion via pump during radiation with radiotherapy such as 1.8 Gy/day to a total of 50.4 Gy. Optionally, a chemotherapeutic agent may be administered to the patient prior to or following the administrations described above, including, but not limited to, capecitabine for a period of time, such as 1 to 6 weeks.

In an example, the present invention may be provided as a neoadjuvant for locally advanced, unresectable disease and may comprise, for example, in each cycle of treatment, administering a first pharmaceutical composition comprising SapC-DOPS and a second pharmaceutical composition comprising gemcitabine or 5′-fluorouracil to a patient, either sequentially or contemporaneously. For example, each cycle may comprise at least once weekly administration of the first composition comprising SapC-DOPS at a dose of 2.4 mg/kg and sequential or contemporaneous administration of the second composition comprising gemcitabine at a dose of 1000 mg/m². Alternatively, each cycle may comprise, on a 31-day treatment regime, administering the first composition comprising SapC-DOPS at least once per week and sequential or contemporaneous administration of the second composition comprising at a bolus dose of 5′-fluorouracil, such as 500 mg/m²/day IV on days 1-3 and days 29-31, with concurrent radiotherapy, such as 40 Gy.

In an example, the present invention may be provided as a first-line treatment for metastatic disease and may comprise, for example, in each cycle of treatment, administering a first pharmaceutical composition comprising SapC-DOPS, a second pharmaceutical composition comprising nab-paclitaxel, and a third pharmaceutical composition comprising gemcitabine to a patient, either sequentially or contemporaneously. For example, each cycle may comprise weekly administration of the first composition comprising SapC-DOPS at a dose of 2.4 mg/kg at least once per week, such as three times per week, and sequential or contemporaneous administration of the second composition comprising nab-paclitaxel at a dose of 100-125 mg/m² and sequential or contemporaneous administration of the third composition comprising gemcitabine at a dose of 1000 mg/m² IV on days 1, 8, and 15 of each 28 day cycle. For example, the administration schedule of SapC-DOPS plus gemcitabine plus nab-paclitaxel shown in Tables 1 and 2 may be followed according to the present invention.

TABLE 1
Administration Schedule of SapC-DOPS
On Days of infusion by IV Infusion over 45 min ± 15 min

| Week 1 | Week 2 | Week 3 | Week 4 |
|---|---|---|---|
| 3x/week | 3x/week | Once on day 15 | Off week |
| (Every other | (Every other | | |
| day) | day) | | |

TABLE 2
Administration Schedule of nab-paclitaxel follow by
gemcitabine
Day 1, day 8, and day 15 over 30 min-40 min

| Week 1 | Week 2 | Week 3 | Week 4 |
|---|---|---|---|
| Day 1 | Day 8 | Day 15 | Off week |

In an example, the present invention may be provided as a first-line treatment for metastatic disease and may comprise, for example, in each cycle of treatment, administering a first pharmaceutical composition comprising SapC-DOPS and a second pharmaceutical composition comprising gemcitabine to a patient, either sequentially or contemporaneously. For example, each cycle may comprise weekly administration of the first composition comprising SapC-DOPS at a dose of 2.4 to 5.0 mg/kg and sequential or contemporaneous administration of the second composition comprising gemcitabine at a dose of 1000 mg/m² weekly for 7 weeks, followed by 1 week off, followed by weekly for 3 weeks.

In an example, the present invention may be provided as a first-line treatment for metastatic disease and may comprise, for example, in each cycle of treatment, administering a first pharmaceutical composition comprising SapC-DOPS, a second pharmaceutical composition comprising gemcitabine, and a third pharmaceutical composition comprising cisplatin to a patient, either sequentially or contemporaneously. For example, the method may comprise, administration of the first composition comprising SapC-DOPS at least once per week and sequential or contemporaneous administration of the second composition comprising gemcitabine at a dose of 1000 mg/m² and sequential or contemporaneous administration of the third composition comprising cisplatin at a dose of 50 mg/m² on days 1 and 15 of a 28 day cycle.

In an example, the present invention may be provided as a first-line treatment for metastatic disease and may comprise, for example, in each cycle of treatment, administering a first pharmaceutical composition comprising SapC-DOPS, a second pharmaceutical composition comprising gemcitabine, and a third pharmaceutical composition comprising erlotinib to a patient, either sequentially or contemporaneously. For example, the method may comprise, daily on days 1-56, administration of the first composition comprising SapC-DOPS at a dose of 2.4-5 mg/kg and sequential or contemporaneous administration of the second composition comprising gemcitabine at weekly dose of 1000 mg/m² and sequential or contemporaneous administration of the third composition comprising erlotinib at a weekly dose (100 mg PO daily), for up to 4 cycles.

In an example, the present invention may be provided as a first-line treatment for metastatic disease and may comprise, for example, in each cycle of treatment, administering a first pharmaceutical composition comprising SapC-DOPS, a second pharmaceutical composition comprising gemcitabine, and a third pharmaceutical composition comprising capecitabine to a patient, either sequentially or contemporaneously. For example, the method may comprise, administration of the first composition comprising SapC-DOPS at a dose of 2.4-5.0 mg/kg and sequential or contemporaneous administration of the second composition comprising gemcitabine at a dose of 1000 mg/m² and sequential or contemporaneous administration of the third composition comprising capecitabine at a dose of 1660 mg/m²/day.

In an example, the present invention may be provided as a first-line treatment for metastatic disease and may comprise, for example, in each cycle of treatment, administering a first pharmaceutical composition comprising SapC-DOPS and a second pharmaceutical composition comprising Folfirinox, either sequentially or contemporaneously. For example, the method may comprise administration of the first composition comprising SapC at a dose of 2.4-5.0 mg/kg and sequential or contemporaneous administration of the second pharmaceutical composition comprising FOLFIRINOX (Oxaliplatin 85 mg/m² IV on day 1 plus irinotecan180 mg/m² IV on day 1 plus leucovorin 400 mg/m² IV on day 1, followed by 5-FU 400 mg/m² IV bolus on day 1 and then 2400 mg/m² IV infusion over 46 h on days 1 and 15).

In an example, the present invention may be provided as a second-line treatment for metastatic pancreatic cancer and may comprise, for example, in each cycle of treatment, administering a first pharmaceutical composition comprising SapC-DOPS and a second pharmaceutical composition comprising capecitabine. For example, the method may comprise administration of the first composition comprising SapC-DOPS at a dose of 2.4 mg/kg and sequential or contemporaneous administration of the second pharmaceutical composition comprising capecitabine at a dose if 1250 mg/m² PO BID for 14 days, every 3 wk. Optionally, the treatment may further comprise sequential or contemporaneous administration of a third pharmaceutical composition comprising erlotinib (150 mg PO daily continuously).

In an example, the present invention may be provided as a second-line treatment for metastatic pancreatic cancer and may comprise, for example, in each cycle of treatment, administering a first pharmaceutical composition comprising SapC-DOPS at a dose of 2.4-5.0 mg/kg at least once per week, such as three times per week, and sequential or contemporaneous administration of a second pharmaceutical composition comprising modified Folfirinox (Oxaliplatin 85 mg/m² IV on day 1 plus irinotecan liposomal, 70 mg/m² IV infused over 90 min, followed by leucovorin 400 mg/m² IV infused over 30 min, followed by fluorouracil 2400 mg/m² IV infused over 46 hours, every 3 weeks).

In an example, the present invention may be provided as a second-line treatment for metastatic pancreatic cancer and may comprise, for example, in each cycle of treatment, administering a first pharmaceutical composition comprising SapC-DOPS at a dose of 2.4-5.0 mg/kg at least once per week and sequential or contemporaneous administration of a second pharmaceutical composition comprising modified Folfirinox (5′fluorouracil 2000 mg/m² IV over 24 hours on days 1, 8, 15, and 22, leucovorin 200 mg/m² IV over 30 minutes on days 1, 8, 15, and 22, and oxaliplatin 85 mg/m² IV on days 8 and 22, every 42 days).

The results of treatment with the methods of the present invention may be evaluated or determined by any method known to those skilled in the art, including, but not limited to, imaging, ultrasounds, physical examination, blood tests, and radioimmunoassays.

Kits

The pharmaceutical compositions described herein may be included in a kit, pack, dispenser for treating a cancer or inhibiting tumor growth, referred to collectively herein as a “kit,” optionally with instructions for administration of such pharmaceutical compositions. A kit may include the first pharmaceutical composition and the second pharmaceutical composition. Optionally, additional pharmaceutical compositions also may be included. The kit also may include a pharmaceutically acceptable carrier suitable for each pharmaceutical composition included therein. The compositions and carrier(s) may be housed in vials or other suitable containers. The compositions may be lyophilized, resuspended, liquid, powder, or in any other suitable form.

The first pharmaceutical composition may comprise the SapC-DOPS composition described hereinabove.

The second, third, fourth, etc. pharmaceutical composition may comprise one or more of the antineoplastic agents described hereinabove, including, but not limited to, gemcitabine, paclitaxel or nab-paclitaxel, or 5′-fluorouracil, including Folfirinox or modified Folfirinox.

The kit may include instructions recorded on any recording medium known to those skilled in the art, and may set forth instructions for reconstituting the pharmaceutical compositions contained in the kit and/or for practicing the method of the invention disclosed herein. Optionally, the instructions may include instructions to download or otherwise access instructions that are remotely stored. In examples, the recording medium may include, but is not limited to, a kit insert, a label on one or more of the containers housing the pharmaceutical compositions or carriers, or may be stored on any computer readable storage medium. The instructions may be stored remotely in downloadable or non-downloadable form, accessible, for example, via the internet.

In view of the foregoing description the present invention thus relates in particular, without being limited thereto, to the following Aspects 1-26:

### ASPECTS

Aspect 1. A method of treating pancreatic cancer comprising administering a first pharmaceutical composition comprising Saposin C and dioleoylphosphatidylserine (SapC-DOPS) and administering a second pharmaceutical composition comprising an antineoplastic agent.

Aspect 2. The method of Aspect 1, wherein the SapC-DOPS is present in nanovesicles.

Aspect 3. The method of Aspect 1 or 2, wherein the antineoplastic agent comprises gemcitabine, nab-paclitaxel, Folfirinox, or a combination thereof.

Aspect 4. The method of any of the preceding Aspects, wherein the first pharmaceutical composition is administered contemporaneously or sequentially with the second pharmaceutical composition.

Aspect 5. The method of any of the preceding Aspects, wherein the administering of the first pharmaceutical composition comprises an intravenous route.

Aspect 6. The method of any of the preceding Aspects, wherein the administering of the second pharmaceutical composition comprises an intravenous route.

Aspect 7. The method of any of the preceding Aspects, wherein the administering of the second pharmaceutical composition results in increased external neoplastic cell membrane surface levels of phosphatidylserine (PS).

Aspect 8. The method of any of the preceding Aspects, further comprising a third pharmaceutical composition.

Aspect 9. The method of Aspect 8, wherein the first pharmaceutical composition comprises SapC-DOPS at a dose of 2.4 mg/kg and the administering of the first pharmaceutical composition occurs at least once per week, the second pharmaceutical composition comprises gemcitabine at a dose of 1000 mg/m², and the third composition comprises nab-paclitaxel at a dose of 100 mg/m².

Aspect 10. The method of Aspect 9, wherein the administering of the first pharmaceutical composition occurs at least three times per week, the administering of the second therapeutic composition occurs one time per week, or combinations thereof.

Aspect 11. A method of inhibiting tumor growth comprising administering a first pharmaceutical composition comprising SapC-DOPS and administering a second pharmaceutical composition comprising a antineoplastic agent.

Aspect 12. The method of Aspect 11, wherein the second pharmaceutical composition comprises gemcitabine, nab-paclitaxel, Folfirinox, or a combination thereof.

Aspect 13. The method of Aspect 11 or 12, wherein the first pharmaceutical composition is administered contemporaneously or sequentially with the second pharmaceutical composition.

Aspect 14. The method of any of Aspects 11-13, further comprising a third pharmaceutical composition.

Aspect 15. The method of Aspect 14, wherein first pharmaceutical composition comprises SapC-DOPS at a dose of 2.4 mg/kg and the administering of the first pharmaceutical composition occurs at least once per week, the second pharmaceutical composition comprises gemcitabine at a dose of 1000 mg/m², and the third composition comprises nab-paclitaxel at a dose of 100 mg/m².

Aspect 16. The method of Aspect 15, wherein the administering of the first pharmaceutical composition occurs at least three times per week.

Aspect 17. A kit for the treatment of pancreatic cancer comprising at least two pharmaceutical compositions, wherein a first pharmaceutical composition comprises SapC-DOPS and wherein a second pharmaceutical composition comprises a first antineoplastic agent.

Aspect 18. The kit of Aspect 17, wherein the kit further comprises instructions for administering the first and the second pharmaceutical compositions.

Aspect 19. The kit of Aspect 17 or 18, wherein the second pharmaceutical composition comprises gemcitabine.

Aspect 20. The kit of any of Aspects 17-19, further comprising a third pharmaceutical composition comprising a second antineoplastic agent, the kit further comprising instructions for administering the third pharmaceutical composition.

Aspect 21. The kit of Aspect 20, wherein the third pharmaceutical composition comprises nab-paclitaxel.

Aspect 22. The kit of Aspect 21, wherein first pharmaceutical composition comprises SapC-DOPS at a dose of 2.4 mg/kg and the instructions instruct administering the first pharmaceutical composition at least once per week, the second pharmaceutical composition comprises gemcitabine at a dose of 1000 mg/m², and the third composition comprises nab-paclitaxel at a dose of 100 mg/m².

Aspect 23. The kit of Aspect 22, wherein the instructions instruct administering the first pharmaceutical composition at least three times per week.

Aspect 24. The kit of Aspect 17 or 18, wherein the second pharmaceutical composition comprises Folfirinox.

Aspect 25. A combination therapeutic comprising a first pharmaceutical composition comprising SapC-DOPS and a second pharmaceutical composition comprising at least one antineoplastic agent, wherein the first and the second pharmaceutical compositions are formulated separately to be used in the form of a kit where they are present together.

Aspect 26. The combination therapeutic of Aspect 25, further comprising a third pharmaceutical composition comprising a second antineoplastic agent formulated separately to be used in the kit.

Illustrating the invention are the following examples that are not to be considered as limiting the invention to their details.

### EXAMPLES

### Example 1

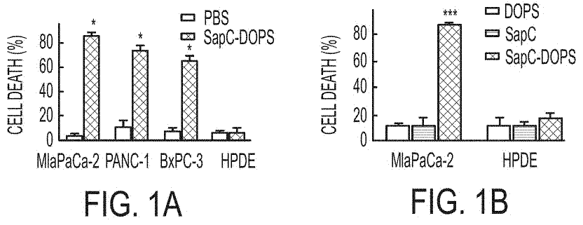

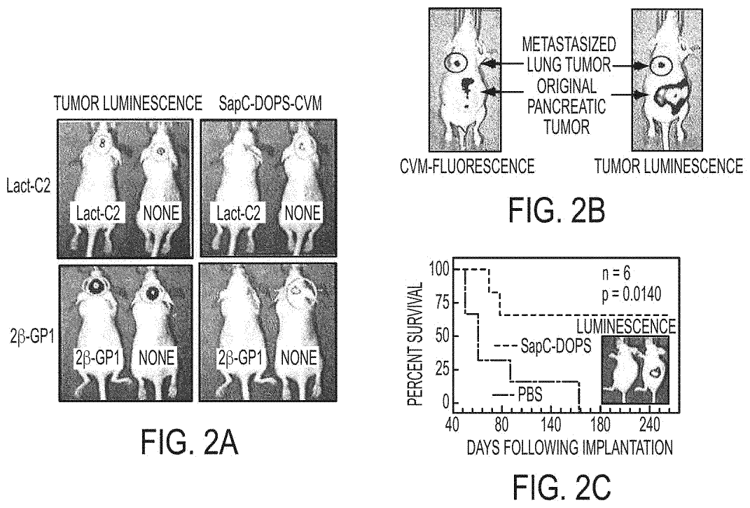

SapC-DOPS has cytotoxic effects against pancreatic cancer cells in vitro and in vivo. As shown in FIG. 1A, exposure to SapC-DOPS led to extensive death in cultured human pancreatic tumor cell lines, but not in non-transformed pancreatic ductal cells. The data demonstrate that cytotoxic action required specific SapC-PS interaction, as DOPS liposomes alone were ineffective (FIG. 1B), and masking PS in cancer cells with beta-glycoprotein or lactadherin greatly diminished SapC-DOPS targeting (FIG. 2A). In an orthotopic mouse model of pancreatic cancer, fluorescently labeled SapC-DOPS (SapC-DOPS-CVM) effectively targeted both the primary tumor and also metastatic foci (FIG. 2B). SapC-DOPS treatment produced significant tumor suppression in heterotopic human pancreatic tumor xenografts, while complete tumor eradication was achieved in orthotopic xenografts (FIG. 2C).

### Example 2

Combination treatment with GEM and SapC-DOPS has synergistic antitumor effects. Most patients with non-resectable pancreatic cancer are treated with GEM, a nucleoside analog that halts DNA replication and, as shown in animal models of pancreatic cancer, induces PS exposure on the surface of pancreatic cancer cells and tumor vasculature. These data demonstrate that the combined use of GEM and SapC-DOPS may be a superior therapeutic option for pancreatic cancer.

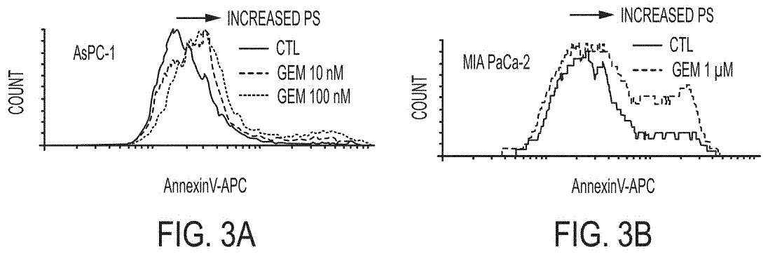

FIG. 3 shows that GEM exposure caused a dose-dependent increase in external PS in viable human pancreatic cancer cell lines with relatively low (AsPC-1) (FIG. 3A) or moderate (MIA-PaCa-2) (FIG. 3B) surface PS levels. Importantly, in low surface PS cells a clear increase was evident using sub-toxic concentrations of GEM that caused <10% apoptosis.

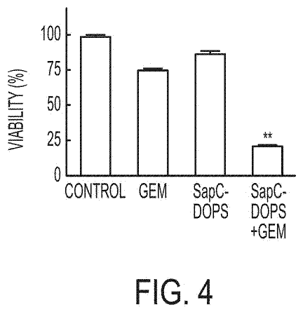

FIG. 4 shows that the drug combination induced synergistic cell death on cultured Mia-Paca-2 cells and completely inhibited tumor growth in established heterotopic xenografts (FIG. 4A-4C), supporting the tenet that enhanced antitumor effects were afforded by combined treatment with SapC-DOPS/GEM Results in vitro and in vivo. These data strongly suggest that the PS-inducing effect of GEM can be exploited to sensitize tumors to SapC-DOPS cytotoxic actions. Co-treatment with GEM and SapC-DOPS may thus be a breakthrough in pancreatic cancer therapy, readily testable in thousands of patients already receiving GEM as first-line treatment.

Methods:

Various cell types were analyzed for surface PS exposure by flow cytometry with annexin V-FTIC. Involvement of flippases in the regulation of surface PS exposure was analyzed by flippase activity assay. Total cellular PS was quantified by TLC separation of phospholipids and estimation of PS phosphorous. Orthotopic PDAC tumors were established by injecting human or murine PDAC cells into animal pancreas. In vivo bioluminescence and fluorescence imaging were performed to monitor tumor targeting and growth. The molecular mechanisms underlying the induction of apoptosis by SapC-DOPS were evaluated in cultured PDAC cells through measurements of cell viability, TUNEL, and flow cytometric DNA fragmentation.

Results:

It was demonstrated in a live animal imaging system that fluorescently labeled SapC-DOPS nanovesicles accumulated in orthotopic PDAC tumors via a PS-selective mechanism. We determined that PS exposure on the surface of PD AC cells was variable. Cancer cells exhibited elevated surface PS and fell into low and high surface PS groups. Our results identify differential flippase activity as one of the major regulators of surface PS exposure among cancer cells. We also observed a correlation between total cellular PS and surface PS exposure, with high surface PS cancer cells having relatively high intracellular calcium and total cellular PS compared to low surface PS cells. Chemotherapy (i.e. gemcitabine) was found to increase surface PS levels on PDAC cells. Enhancement of SapC-DOPS anticancer effect was determined by combining chemotherapy in PDAC cells and tumors. Combination treatment with SapC-DOPS and gemcitabine showed a synergistic effect on PDAC cells and tumors, presumably by enhancing SapC-DOPS anticancer potency. Conclusion: SapC-DOPS nanovesicles had PS-specific targeting activity on PDAC cells in orthotopic tumors. PS was heterogeneously exposed on the surface of PDAC cell lines. PS low and high cancer cell lines exhibited differential flippase activity and differ in total cellular PS. Chemodrug treatments elevated surface PS levels on pancreatic cancer cells.

Such surface PS increase led to enhancement of SapC-DOPS efficacy in vitro and in vivo.

### Example 3

Reagents and Compounds:

SapC-DOPS was received from Bexion Pharmaceuticals, Inc. and were stored at 80° C. until use. Separate aliquoted vials of SapC-DOPS for the 7.3 mg/kg dose were received. Separate aliquoted vials of vehicle were received, which contained DOPS and no SapC. Each vial was resuspended in 1.2 ml of sterile water for injection (Lot #JPJ551, 13/Braun Medical, Inc., Irvine Calif.). Once formulated, SapC-DOPS was delivered at a 10 ml/kg dose volume. SapC-DOPS and vehicle were formulated fresh daily and administered immediately after preparation.

Gemcitabine (Lot #A735891A) was manufactured by Eli Lilly and Co. (Indianapolis, Ind.) and diluted in a 0.9% NaCl solution (B. Braun Medical, Inc., Irvine, Calif., Lot #J0K465) to a concentration of 4 mg/ml to deliver a 40 mg/kg dose at a 10 ml/kg dose volume. All preparations were made fresh prior to their administration.

Cell Culture:

The MIA Paca-2 human pancreas tumor cell line was received from American Type Culture Collection (ATCC, Manassas, Va.). Cultures were maintained in RPMI-1640 (Hyclone, Logan, Utah) supplemented with 5% fetal bovine serum, and housed in a 5% CO2 atmosphere. The cultures were expanded in tissue culture flasks at a 1:4 split ratio until a sufficient amount of cells were harvested.

Animals:

Female NCR nude mice (CrTac:NCR-Foxn1^{nu}) were supplied by Taconic (Germantown, N.Y.). Mice were received at four weeks' of age, 12-15 g in weight. All mice were acclimated for seven days prior to handling. The mice were housed in microisolator cages (Lab Products, Seaford, Del.) and maintained under specific pathogen-free conditions. The mice were fed PicoLab® irradiated mouse chow (Lab Diet, Richmond, Ind.) and autoclaved water was freely available. All procedures were carried out under the institutional guidelines of TGen Drug Development Services Institutional Animal Care and Use Committee (Protocol #09002, Approved February 2009).

MIA PaCa-2 Human Pancreas Tumor Xenograft Model:

Female mice were inoculated subcutaneously in the right flank with 0.1 ml of a 50% RPMI/50% Matrigel™ (BD Biosciences, Bedford, Mass.) mixture containing a suspension of MIA PaCa-2 tumor cells (approximately 5×10⁶ cells/mouse).

Five days following inoculation, tumors were measured using calipers and tumor weight was calculated using the animal study management software, Study Director V. 1.7.54 k (Study Log)¹, Thirty mice with tumor sizes of 105-158 mg were pair-matched into three groups of ten mice each (Day 1). Body weights were recorded when the mice were pair-matched and were taken twice weekly thereafter in conjunction with tumor measurements. Starting on Day 1, SapC-DOPS, vehicle, and gemcitabine were dosed according to Table 3. The study was terminated on Day 35.

TABLE 3
Treatment Regimen

| | | Vehicle | | SapC- |
|---|---|---|---|---|
| | | Control | Gemcitabine | DOPS |
| Group | N | (Q3Dx4) | (Q3Dx4) | (Q3Dx4) |
| 1. Vehicle Control (IV) | 10 | X | | |
| 2. Gemcitabine 40 mg/kg (IP) | 10 | | X | |
| 3. SapC-DOPS 7.3 mg/kg (IV) + | 10 | | X | X |
| Gemcitabine 40 mg/kg (IP)¹ | | | | |

¹Gemcitabine was given 6 hours before SapC-DOPS

On Day 35, tumors were collected from two randomly selected mice per group. Tumors were fixed in a 4% paraformaldehyde (Lot #066297, Fisher Scientific, Fair Lawn, N.J.) in 1×PBS (Phosphate Buffer Solution, Lot #0711006, Ambion, Austin, Tex.) solution for 48 hours at 4° C. Tumors were then transferred to a 30% sucrose (Lot #098K01844, Sigma-Aldrich, St. Louis, Mo.) in 1×PBS solution for 48 hours at 4° C. The tumors were then transferred to a 30% sucrose in 1×PBS solution for 72 hours. The tumors were then frozen in OCT (Optimal Cutting Temperature, Lot #0004348-01, Sakura Finetek, Torrance, Calif.) and stored at −80° until shipment to sponsor. Lungs were collected from four randomly selected mice in Group 3 (SapC-DOPS+gemcitabine). The lungs were fixed in formalin (Lot #4117, Azer Scientific, Morgantown, Pa.) and sent to the Mayo Clinic Histology (Scottsdale, Ariz.) to be paraffin blocked process through H&E. The lungs were analyzed by a pathologist at Mayo Clinic with emphasis on evidence of granuloma on the lungs.

Data and Statistical Analysis:

Mean tumor weights and TGI are reported in Tables 4 and 5 and mean tumor shrinkage are reported in Table 6. Tables 7 and 8 report tumor weight comparisons.

Mean tumor growth inhibition (TGI) was calculated utilizing the following formula (any tumors that regressed were not used in calculations):

$$TGI = \frac{[ 1 - ( χ {Treated}_{( Final )} - χ {Treated}_{( Day )} ) ]}{( χ {Control}_{( Final )} - χ {Control}_{( Day 1 )}} \times 100 \%$$

Individual tumor shrinkage (TS) was calculated using the formula below for tumors that showed regression relative to Day 1 tumor weight. The mean tumor shrinkage of each group was calculated and reported.

TS=[1−(Tumor Weight_{(final)})/Tumor Weight_{(Day 1)})]×100%

TGI calculations were performed on Days 3-35. All statistical analyses in the xenograft study were performed with GraphPad Prism® v4 software. Differences in tumor weights (Days 3-35) were confirmed using an unpaired two-tailed t-test with Welch's correction.

Mia-Paca-2 Human Pancreas Tumor Xenograft Model:

The vehicle control group reached a mean tumor weight of 958.3 mg on Day 35. One tumor had an overall spontaneous regression. This tumor was not included in any efficacy calculations. No appreciable weight loss was observed during the study.

Treatment with gemcitabine 40 mg/kg resulted in a mean tumor weight of 936.0 mg on. Day 35. This group produced a maximum TGI of 41.6% on Day 14 when compared to vehicle control. A significant difference in tumor weight was observed when compared to the vehicle control on Days 7 (P<0.05), 10 (P<0.05), and 14 (P<0.05). This group experienced tumor shrinkage on Day 3 (n=5, mean TS=11.6%), Day 7 (n=3, mean TS=11.6%), Day 10 (n=2, mean TS=9.6%), Day 14 (n=1, 7.6%). No appreciable weight loss was observed during the study.

Treatment with SapC-DOPS 7.3 mg/kg gemcitabine 40 mg/kg resulted in a mean tumor weight of 366.5 mg on Day 35. This group produced a maximum TGI of72.0% on Day 30 when compared to vehicle control. A significant difference in tumor weight was observed when compared to the vehicle control on Days 7 (P<0.005), 10 (P<0,005), 14 (P<0.005), 17 (P<0.05), 20 (P<0.05), 23(P<0.05), 27 (P<0.05), 30 (P<0.05), and 35 (P<0.05). This group produced a maximum TGI of 65.0% on Day 30 when compared to gemcitabine as a single agent. A significant difference in tumor weight was observed when compared to gemcitabine as a single agent on Days 23 (P<0.05), 27 (P<0.01), 30 (P<0.005), and 35 (P<0.005). This group experienced tumor shrinkage on Day 3 (n=2, mean TS=8.2%), Day 7 (n=3, mean TS=13.7%), Day 10 (n=2, mean TS=16.6%), Day 14 (n=3, mean TS=11.5%), Day 20 (n=3, mean TS=27.2%), Day 23 (n=1, 5.4%), Day 27 (n=1, 56.1%), Day 30 (n=1, 46.1%), and Day 35 (n==4, mean TS=47.7%). This group experienced no weight loss throughout the study, No granulomas were observed on the lungs upon examination by pathologist.

### Example 4

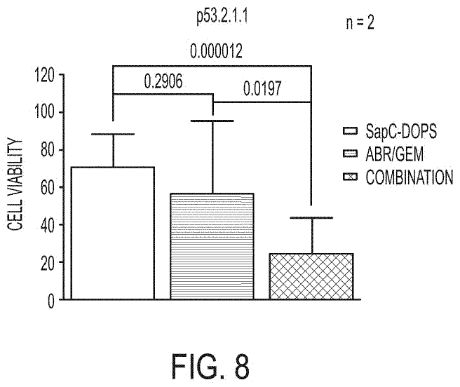

Mouse pancreatic cancer cells (p53.2.1.1) were plated in 96 wells overnight. The following day they were treated with either 20 μM SapC-DOPS, 25 nMNab-paclitaxel/abraxane(Abr)-25 nM gemcitabine (Abr/GEM) or a combination of SapC-DOPS and Abr/GEM. Untreated cells were used to determine 100% cell viability by MTT assay. The means and standard deviations of 2 experiments with 6 data points for each experiment are shown in FIG. 8, demonstrating an improved cell viability of mouse pancreatic cancer cells following treatment with a combination of SapC-DOPS (20 μM) plus 25 nM Abraxane® plus 25 nM Gemcitibine compared to treatment with either SapC-DOPS (20 μM) or 25 nM Abraxane® plus 25 nM Gemcitibine alone.

TABLE 4
Day 35 Mean Tumor Weights and TGI (Days 3-35)-All Groups vs Vehicle Control

| | | | Day 35 | | | | | |
|---|---|---|---|---|---|---|---|---|
| | | | TW (mg) ± | Day 3 | Day 7 | Day 10 | Day 14 | Day 17 |
| Group | N | Dose | SEM | % TGI (n) | % TGI (n) | % TGI (n) | % TGI (n) | % TGI (n) |
| Vehicle Control | 10 | — | 958.3 ± 193.8 | — | — | — | — | — |
| Gemcitabine | 10 | 40 mg/kg | 936.0 ± 135.1 | 16.3 (5) | 39.5 (7) | 37.1 (8) | 41.6 (9) | 40.1 (10) |
| SapC-DOPS + | 10 | 7.3 mg/kg | 366.5 ± 109.2 | NR (8) | 58.6 (7) | 55.8 (8) | 59.6 (7) | 60.3 (10) |
| Gemcitabine | | 40 mg/kg | | | | | | |

| | Day 20 | Day 23 | Day 27 | Day 30 | Day 35 | |
|---|---|---|---|---|---|---|
| Group | % TGI (n) | % TGI (n) | % TGI (n) | % TGI (n) | % TGI (n) | Total Deaths |
| Vehicle Control | — | — | — | — | — | 0 |
| Gemcitabine | 31.1 (10) | 30.3 (10) | 7.7 (10) | 20.0 (10) | 12.8 (10) | 0 |
| SapC-DOPS + | 47.5 (7) | 58.0 (9) | 56.8 (9) | 72.0 (9) | 52.6 (6) | 0 |
| Gemcitabine | | | | | | |

TW = Tumor Weight
NR = No reportable TGI
(n) = number of animals considered in analysis

TABLE 5
TGI (Days 3-35)-TGI (Days 3-35) Combination vs Gemcitabine

| | | | Day 3 | Day 7 | Day 10 | Day 14 | Day 17 | Day 20 |
|---|---|---|---|---|---|---|---|---|
| Group | N | Dose | % TGI (n) | % TGI (n) | % TGI (n) | % TGI (n) | % TGI (n) | % TGI (n) |
| SapC-DOPS + | 10 | 7.3 mg/kg | NR (8) | 31.6 (7) | 29.7 (8) | 30.8 (7) | 31.8 (10) | 23.8 (7) |
| Gemcitabine | | 40 mg/kg | | | | | | |

| | | | | Day 35 | Total |
|---|---|---|---|---|---|
| Group | Day 23 % TGI (n) | Day 27 % TGI (n) | Day 30 % TGI (n) | % TGI (n) | Deaths |
| SapC-DOPS + | 39.8 (9) | 53.3 (9) | 65.0 (9) | 45.6 (6) | 0 |
| Gemcitabine | | | | | |

NR = No reportable TGI
(n) = number of animals considered in analysis

TABLE 6
Days 3-35 Mean Tumor Shrinkage

| | | | Day 3 | Day 7 | Day 10 | Day 14 | Day 17 | Day 20 |
|---|---|---|---|---|---|---|---|---|
| Group | n | Dose | % TS (n) | % TS (n) | % TS (n) | % TS (n) | % TS (n) | % TS (n) |
| Vehicle Control | 10 | — | 11.7 (1) | 6.1 (1) | — | — | — | — |
| Gemcitabine | 10 | 40 mg/kg | 11.6 (5) | 11.6 (3) | 9.6 (2) | 7.6 (1) | — | — |
| SapC-DOPS + | 10 | 7.3 mg/kg | 8.2 (2) | 13.7 (3) | 16.6 (2) | 11.5 (3) | — | 27.2 (3) |
| Gemcitabine | | 40 mg/kg | | | | | | |

| Group | Day 23 % TS (n) | Day 27 % TS (n) | Day 30 % TS (n) | Day 35 % TS (n) |
|---|---|---|---|---|
| Vehicle Control | — | — | 14.8 (1) | 41.7 (1) |
| Gemcitabine | — | — | — | — |
| SapC-DOPS + | 5.4 (1) | 56.1 (1) | 46.1 (3) | 47.7 (4) |
| Gemcitabine | | | | |

TS = Tumor Shrinkage
(n) = number of animals considered in analysis

TABLE 7
Days 3-35 Tumor Weight Comparisons: All Groups vs. Vehicle Control

| | | Day 3 | Day 7 | Day 10 | Day 14 | Day 17 |
|---|---|---|---|---|---|---|
| Group | Comparison | Outcome | Outcome | Outcome | Outcome | Outcome |
| Gemcitabine | Vehicle | NS | P < 0.05 | P < 0.05 | NS | NS |
| 40 mg/kg | | | | | | |
| SapC-DOPS | Vehicle | NS | P < 0.005 | P < 0.005 | P < 0.005 | P < 0.05 |
| 7.3 mg/kg + | | | | | | |
| Gemcitabine | | | | | | |
| 40 mg/kg | | | | | | |

| | Day 20 | Day 23 | Day 27 | Day 30 | |
|---|---|---|---|---|---|
| Group | Outcome | Outcome | Outcome | Outcome | Day 35 Outcome |
| Gemcitabine | NS | NS | NS | NS | NS |
| 40 mg/kg | | | | | |
| SapC-DOPS | P < 0.05 | P < 0.05 | P < 0.05 | P < 0.05 | P < 0.05 |
| 7.3 mg/kg + | | | | | |
| Gemcitabine | | | | | |
| 40 mg/kg | | | | | |

NS = Not Significant

TABLE 8
Days 3-35 Tumor Weight Comparisons: Combination vs Gemcitabine

| | | Day 3 | Day 7 | Day 10 | Day 14 | Day 17 |
|---|---|---|---|---|---|---|
| Group | Comparison | Outcome | Outcome | Outcome | Outcome | Outcome |
| SapC-DOPS | Gemcitabine | NS | NS | NS | NS | NS |
| 7.3 mg/kg + | 40 mg/kg | | | | | |
| Gemcitabine | | | | | | |
| 40 mg/kg | | | | | | |

| | Day 20 | Day 23 | Day 27 | Day 30 | |
|---|---|---|---|---|---|
| Group | Outcome | Outcome | Outcome | Outcome | Day 35 Outcome |
| SapC-DOPS | NS | P < 0.05 | P < 0.01 | P < 0.005 | P < 0.005 |
| 7.3 mg/kg + | | | | | |
| Gemcitabine | | | | | |
| 40 mg/kg | | | | | |

NS = Not Significant

A study of the antitumor effects of gemcitabine as a single agent and SapC-DOPS and gemcitabine in combination against MIA PaCa-2 human pancreas tumor xenograft was completed. Efficacy was assessed by tumor growth inhibition and statistical comparisons of tumor weights to vehicle control group on Days 3-35.

The combination of SapC-DOPS and gemcitabine exhibited a significant decrease in tumor weight when compared to the vehicle control on all days except for Day 3. This combination group showed an overall decrease in tumor weight beginning on Day 7 when compared to gemcitabine alone, and statistically significant differences beginning on Day 23 and continuing through the end of the study (Day 35).

All test agents (SapC-DOPS and gemcitabine) were well tolerated throughout the study. No adverse reactions to dosing were observed at any point during the study. No appreciable weight loss resulted in any group throughout the study.

Whereas particular features of the present invention have been described above for purposes of illustration, it will be evident to those skilled in the art that numerous variations of the details of the coating composition, coating, and methods disclosed herein may be made without departing from the scope in the appended claims.

We claim:

1. A method of treating pancreatic cancer comprising administering a first pharmaceutical composition comprising Saposin C and dioleoylphosphatidylserine (SapC-DOPS) and administering a second pharmaceutical composition comprising an antineoplastic agent.

2. The method of claim 1, wherein the SapC-DOPS is present in nanovesicles.

3. The method of claim 1, wherein the antineoplastic agent comprises gemcitabine, nab-paclitaxel, Folfirinox, or a combination thereof.

4. The method of claim 1, wherein the first pharmaceutical composition is administered contemporaneously or sequentially with the second pharmaceutical composition.

5. The method of claim 1, wherein the administering of the first pharmaceutical composition comprises an intravenous route.

6. The method of claim 1, wherein the administering of the second pharmaceutical composition comprises an intravenous route.

7. The method of claim 1, wherein the administering of the second pharmaceutical composition results in increased external neoplastic cell membrane surface levels of phosphatidylserine (PS).

8. The method of claim 1, further comprising a third pharmaceutical composition.

9. The method of claim 8, wherein the first pharmaceutical composition comprises SapC-DOPS at a dose of 2.4 mg/kg and the administering of the first pharmaceutical composition occurs at least once per week, the second pharmaceutical composition comprises gemcitabine at a dose of 1000 mg/m², and the third composition comprises nab-paclitaxel at a dose of 100 mg/m².

10. The method of claim 9, wherein the administering of the first pharmaceutical composition occurs at least three times per week, the administering of the second pharmaceutical composition occurs one time per week, or combinations thereof.

11. A method of inhibiting tumor growth comprising administering a first pharmaceutical composition comprising SapC-DOPS and administering a second pharmaceutical composition comprising an antineoplastic agent.

12. The method of claim 11, wherein the second pharmaceutical composition comprises gemcitabine, nab-paclitaxel, Folfirinox, or a combination thereof.

13. The method of claim 11, wherein the first pharmaceutical composition is administered contemporaneously or sequentially with the second pharmaceutical composition.

14. The method of claim 11, further comprising a third pharmaceutical composition.

15. The method of claim 14, wherein first pharmaceutical composition comprises SapC-DOPS at a dose of 2.4 mg/kg and the administering of the first pharmaceutical composition occurs at least once per week, the second pharmaceutical composition comprises gemcitabine at a dose of 1000 mg/m², and the third composition comprises nab-paclitaxel at a dose of 100 mg/m².

16. The method of claim 15, wherein the administering of the first pharmaceutical composition occurs at least three times per week.

17. A kit for the treatment of pancreatic cancer comprising at least two pharmaceutical compositions, wherein a first pharmaceutical composition comprises SapC-DOPS and wherein a second pharmaceutical composition comprises a first antineoplastic agent.

18. The kit of claim 17, wherein the kit further comprises instructions for administering the first and the second pharmaceutical compositions.

19. The kit of claim 17, wherein the second pharmaceutical composition comprises gemcitabine.

20. The kit of claim 17, further comprising a third pharmaceutical composition comprising a second antineoplastic agent, the kit further comprising instructions for administering the third pharmaceutical composition.

21. The kit of claim 20, wherein the third pharmaceutical composition comprises nab-paclitaxel.

22. The kit of claim 20, wherein first pharmaceutical composition comprises SapC-DOPS at a dose of 2.4 mg/kg and the instructions instruct administering the first pharmaceutical composition at least once per week, the second pharmaceutical composition comprises gemcitabine at a dose of 1000 mg/m², and the third composition comprises nab-paclitaxel at a dose of 100 mg/m².

23. The method of claim 22, wherein the instructions instruct administering the first pharmaceutical composition at least three times per week.

24. The kit of claim 17, wherein the second pharmaceutical composition comprises Folfirinox.

25. The kit of claim 24, wherein the instructions instruct administering Folfirinox intravenously as an infusion.

26. A combination therapeutic comprising a first pharmaceutical composition comprising SapC-DOPS and a second pharmaceutical composition comprising at least one antineoplastic agent, wherein the first and the second pharmaceutical compositions are formulated separately to be used in the form of a kit where they are present together.

27. The combination therapeutic of claim 26, further comprising a third pharmaceutical composition comprising a second antineoplastic agent formulated separately to be used in the kit.


SEQUENCE LISTING (1 sequence)

SEQ ID NO: 1 - protein, 80 aa, Homo sapiens
SDVYCEVCEFLVKEVTKLIDNNKTEKEILDAFDKMCSKLPKSLSEECQEVVDTYGSSILSILLEEVSPELVCSMLHLCSG